(19) United States
(12) Patent Application Publication
(10) Pub. No.: US 2006/0210635 A1
(43) Pub. Date: Sep. 21, 2006

(54) Injectable gel-forming compositions based on cross-linked and non-cross-linked polymers and the use thereof

(75) Inventors: Alexandre Laurent, Courbevoie (FR); Denis Labarre, Villebon (FR); Jiri Labsky, Praha (CZ); Jiri Honiger, Villejuif (FR); Rene Chapot, Limoges (FR); Michel Wassef, Paris (FR); Aymeric Seron, Paris (FR)

Correspondence Address: C. IRVIN MCCLELLAND;OBLON, SPIVAK, MCCLELLAND, MAIER & NEUSTADT, P.C., 1940 DUKE STREET, ALEXANDRIA, VA, 22314, US

(73) Assignees: ASSISTANCE PUBLIQUE-HOPITAUX DE PARIS, Paris (FR); UNIVERSITE PARIS SUD XI, Orsay (FR)

(21) Appl. No.: 10/541,362

(22) PCT Filed: Jan. 5, 2004
(86) PCT No.: PCT/FR04/00003

§ 371 Date: Apr. 13, 2006

(30) Foreign Application Priority Data
Jan. 6, 2003 (FR) 0300067

(51) Int. Cl. A61K 9/14

(52) U.S. Cl. 424/486; 424/487; 424/488

(57) ABSTRACT

The invention relates to injectable gel-forming compositions which contain an association of linear and cross-linked polymers and are embodied in the form of a suspension. The use of said compositions, in particular for filling pipes and cavities is also disclosed.

The present invention relates to injectable gel-forming compositions containing a combination of linear polymers and of crosslinked polymers, and also to the uses thereof, in particular for producing therapeutic occlusions (embolizations), filling pipes and cavities, or percutaneous implantation.

The use of resorbable or nonresorbable biomaterials is common in the medical field. These biomaterials may be in various forms and may be used, for example, for producing therapeutic vascular occlusions (embolizations), for tissue reconstruction, the treatment of gastro-esophageal reflux or of urinary incontinence, percutaneous implantation, or the reduction of wrinkles and, in general, for filling pipes and cavities.

In this context, it is common practice to use injectable materials which are in gelled form and which solidify in situ just after injection.

Thus, the injectable acrylic adhesives and cements, for example based on isobutyl cyanoacrylate, on N-butyl cyanoacrylate or else on poly(methyl methacrylate) (PMMA), have already been proposed. However, these materials have the drawback of being toxic and difficult to handle since they can lead to coating of the catheter used for the injection. It is also difficult to control the in situ polymerization time of these materials.

It has also been proposed, in particular in U.S. Pat. Nos. 5,580,568, 5,695,480 and 5,851,508, to replace the acrylic adhesives and cements with gel forming solutions composed mainly of a water-insoluble polymer in solution in a biocompatible and water-miscible solvent such as, for example, dimethyl sulfoxide (DMSO). According to these documents, the polymer in solution solidifies in situ after injection, according to a phenomenon of extraction of the solvent which is water-miscible and, consequently, miscible with the naturally aqueous physiological medium.

However, these gel-forming solutions have a certain number of drawbacks:
the time required for the departure of the solvent conditions the gelling time of the polymer. Thus, the greater the amount of solvent present in the solution, the longer the gelling time;
the solution in the process of gelling in situ only has a weak mechanical strength against the circulating stream. It is the outer layer in contact with the blood that gels first, while the core of the deposit remains liquid. The deformability of the deposit under the stream remains considerable as long as the gelling phase of the polymer is not complete. This time may be too long for the polymer to solidify before the solution has been carried away by the bloodstream. In practice, the use of such gel-forming solutions means that the injection must be very slow in order for the solution to be able to gel in situ as it leaves the catheter. If the injection is not carried out sufficiently slowly, the gel-forming solution distorts and is either stretched and carried away by the bloodstream as a viscous liquid, or flattened against the vascular wall as a more or less thick layer;
during the solidification of the polymer, a decrease in volume of the order of 20 to 80% takes place (withdrawal phenomenon), which is due to extraction of the solvent by the circulating stream and which is barely compensated for by entry of the physiological liquid into the polymer undergoing solidification (precipitation), which results in the amount of polymer deposited being less than the amount of solution injected. It is not possible to compensate for this loss of volume by an increase in the concentration of polymer within the gel-forming solution since this would lead to a considerable increase in the viscosity of the gel-forming solution, which would hinder or would prevent injection through needles or microcatheters, for example;
the water-miscible, biocompatible solvents used for solubilizing the polymers, in particular DMSO, have considerable local and systemic vascular toxicity (Mottu F et al., PDA J. Pharm. Sci. Technol., 2000, 54(6), 456-469), which is obviously proportional to the dose released into the bloodstream. In addition, with such a solvent, injection of the gel-forming solutions requires the use of special catheters, designed to withstand the solvent. However, even in this case, the solvent can nevertheless damage the microcatheters by means of which it is injected.

In order to remedy all these problems, the inventors have developed what forms the subject of the invention.

A subject of the present invention is therefore a polymer-based injectable gel-forming composition for intratissue and/or intravascular implantation, characterized in that it comprises:
at least one linear polymer that is water-insoluble and soluble in at least one water-miscible solvent,
at least one water-insoluble, hydrophilic crosslinked polymer, said crosslinked polymer having an affinity for said linear polymer, and
at least one biocompatible, water-miscible solvent;
   and in that it is in the form of a suspension of particles of said hydrophilic crosslinked polymer in a solution of said linear polymer.

The injectable gel-forming composition in accordance with the invention has the following physicochemical advantages and characteristics:
it makes it possible to obtain, for the same volume of gel-forming composition injected as with the gel-forming solutions described in the prior state of the art, a final amount of deposited polymer that is comparatively greater per unit volume injected;
it has a viscosity less that that of the injectable solutions described in the prior state of the art containing the same mass of soluble polymer;
it results in greater cohesion of the gel deposited in the vascular lumen;
it makes it possible to decrease the amount of solvent injected into the patient;
it makes it possible to shorten the solidification time of the linear polymer compared with a conventional gel-forming solution, because, since the amount of solvent is less, the kinetics of departure of the solvent from the mixture are more rapid.

When the injectable gel-forming composition in accordance with the invention is injected into a physiological liquid, which by nature is aqueous, the solvent leaves the blend of polymers, resulting in precipitation and solidification of the linear polymer, which then traps the hydrophilic crosslinked polymer. The total amount and the volume of material are increased by the presence of the hydrophilic crosslinked polymer which, due to its affinity for water, swells in an aqueous medium.

For the purpose of the present invention, the term “affinity” used to describe the crosslinked polymer present in the injectable gel-forming composition is understood to mean any cause which prompts the crosslinked polymer to combine with the linear polymer and which keeps them together when the combination is produced. By way of example, this affinity may in particular be chemical.

According to the invention, the linear polymer(s) is (are) preferably chosen from neutral or relatively uncharged polymers.

Among such polymers, mention may in particular be made of poly(alkyl acrylates), poly(alkyl methacrylates), poly(alkyl cyanoacrylates), poly(vinyl acetates), poly(vinyl butyrates), poly(vinyl formals), poly(vinyl acetals), poly(vinyl butyrals), polyoxypropylenes, polyoxytetramethylenes, water-insoluble cellulose esters, water-insoluble esters of chitosan or other polysaccharides, polylactides, polyglycolides, polycaprolactone, poly(malic acid) esters, poly(maleic acid) esters, poly(fumaric acid) esters, and water-insoluble linear copolymers or derivatives comprising these compounds.

Among these polymers, mention may most particularly be made of poly(hydroxyethyl methacrylate) (p(HEMA)), poly(methyl methacrylate) (PMMA), poly(hydroxypropyl methacrylate) (p(HPMA)), copolymers of hydroxyethyl methacrylate or hydroxypropyl methacrylate and of acrylonitrile (HEMA-AN or HPMA-AN), copolymers of hydroxyethyl methacrylate or hydroxypropyl methacrylate and of N-tert-butylacrylamide (HEMA-TBA or HPMA-TBA), copolymers of hydroxyethyl methacrylate or hydroxy-propyl methacrylate and of acetoacetoxyethyl methacrylate (HEMA-AAMA or HPMA-AAMA), poly(N-acryloyl-2-amino-2-hydroxymethyl-1,3-propanediol) such as the product sold under the trade name Trisacryl® by the company Biosepra (France), poly(n-2-hydroxypropyl methacrylamide), and derivatives thereof.

According to a preferred embodiment of the invention, the linear polymer(s) is (are) preferably chosen from copolymers of hydroxypropyl methacrylate and of acrylonitrile (HPMA-AN), copolymers of hydroxypropyl methacrylate and of N-tert-butylacrylamide (HPMA-TBA) and copolymers of hydroxypropyl methacrylate and of acetoacetoxyethyl methacrylate (HPMA-AAMA).

The linear polymer(s) preferably represent(s) from 3 to 25% (m/V) of the injectable gel-forming composition in accordance with the invention, and even more preferably from 5 to 20% (m/V).

According to the invention, the hydrophilic crosslinked polymer(s) can in particular be chosen from the polymers derived from the crosslinking of the water-insoluble linear polymers as described above.

The hydrophilic crosslinked polymer(s) can also be chosen from the polymers derived from the crosslinking of water-soluble linear polymers, such as alginates; starch derivatives; cellulose ethers; cellulose acetates with a degree of substitution of between 0.6 and 0.8; cellulose sulfates; water-soluble polysaccharides such as dextrans; chitosan salts; acrylic and methacrylic polymers; substituted or unsubstituted polyacrylamides and polymethacrylamides; hydrolyzed derivatives of poly(vinyl acetates), such as poly(vinyl alcohols); polymers derived from polyoxyethylene, polyethyleneimine; soluble salts of polyvinylpyridine; polyvinylpyrrolidone; polyurethanes; salts thereof and copolymers thereof.

Among the crosslinked polymers, mention may most particularly be made of the crosslinked polymers of HEMA, of HPMA or of poly(N-acryloyl-2-amino-2-hydroxymethyl-1,3-propanediol), and also the cross-linked copolymers of HEMA and of poly(N-acryloyl-2-amino-2-hydroxymethyl-1,3-propanediol), or of HPMA and of poly(N-acryloyl-2-amino-2-hydroxymethyl-1,3-propane-diol).

The crosslinking of the polymers can be carried out conventionally according to any method known to those skilled in the art, using a crosslinking agent such as, for example, methylenebisacrylamide.

The degree of crosslinking of the crosslinked polymer is preferably between 0.5 and 12% (m/V), and even more preferably between 1 and 5% (m/V).

The crosslinked polymer(s) preferably represent(s) from 1 to 30% (m/V) of the injectable gel-forming composition in accordance with the invention, and even more preferably from 8 to 12% (m/V).

According to the invention, the size of the particles of crosslinked polymer can range between 1 and 1000 μm, and preferably between 20 and 100 μm.

According to a preferred embodiment of the invention, the injectable gel-forming composition comprises a linear polymer and a crosslinked polymer of the same nature, in the form of particles. By way of example, the injectable gel-forming composition in accordance with the invention can contain at least one linear HEMA or HPMA polymer or a linear HEMA-based or HPMA-based copolymer and particles of crosslinked polymers of HEMA, of HPMA or of poly(N-acryloyl-2-amino-2-hydroxymethyl-1,3-propanediol), and/or of crosslnked copolymers of HEMA and of poly(N-acryloyl-2-amino-2-hydroxymethyl-1,3-propanediol) or of HPMA and of poly(N-acryloyl-2-amino-2-hydroxymethyl-1,3-propanediol).

An injectable gel-forming composition that is particularly preferred according to the invention comprises:
at least one linear, HEMA-based or HPMA-based copolymer, and
at least particles of crosslinked copolymers of HEMA or HPMA and of poly(N-acryloyl-2-amino-2-hydroxy-methyl-1,3-propanediol).

The biocompatible, water-miscible solvent(s) is (are) preferably chosen from weakly viscous organic solvents in order to allow their administration by means of needles or catheters with a diameter of less than or equal to a millimeter.

According to the invention, the term “biocompatible” is intended to mean any solvent that is compatible from a pharmaceutical point of view (Food and Drug Administration (FDA), ISO Standard 10993-13 (1998)) for injection into humans or animals.

Among such solvents, mention may in particular be made of N-methylpyrrolidone, dimethylethylamide, diethylene glycol dimethyl ether (Diglyme®), ethyl lactate, ethanol, dimethoxyethane, DMSO, glycofurol, and mixtures thereof; ethanol and N-methylpyrrolidone being particularly preferred.

According to a particular embodiment of the invention, and when DMSO is used as solvent, the latter is then preferably used in minimal amounts or in combination, as a minor constituent, with one of the other solvents listed above.

The injectable gel-forming compositions in accordance with the invention can be prepared by dissolving the desired amount of linear polymer(s) in the water-miscible solvent, to which the desired amount of crosslinked polymer(s), preferably prepared beforehand in the form of particles (microparticles), is then added. The blends are preferably prepared with magnetic stirring in order in particular to facilitate the homogeneous distribution of the crosslinked polymer(s) in the solution of linear polymer(s).

In the course of this preparation, the linear copolymer-based intermediate solutions comprising at least one linear copolymer of HPMA and of AN, and/or at least one copolymer of HPMA and of TBA, and/or at least one copolymer of HPMA and of AAMA, and at least one biocompatible, water-miscible solvent as, for example, described above are novel per se and constitute, in this respect, another subject of the invention, it being understood that, when said intermediate solution contains a linear copolymer of hydroxypropyl methacrylate and of acrylonitrile, then the solvent is not DMSO.

The injectable gel-forming compositions in accordance with the invention can also contain one or more adjuvants chosen from dyes (in order to make the composition visible when it is injected); imaging markers, such as contrast agents for X-ray imaging, for instance iodinated products and metal powders, including tantalum and tungsten, or for ultrasound or MRI imaging, diagnostic or therapeutic, radioactive products; anti-inflammatory agents; angiogenic agents; antimitotics; angiogenesis inhibitors; growth factors; vitamins; hormones; proteins; vaccines; peptides; antiseptics; antimicrobial agents such as antibiotics; and, in general, any agent for therapeutic, preventive or diagnostic purposes.

According to their solubility with respect to the biocompatible solvent used, these adjuvants can be incorporated into the injectable gel-forming composition in accordance with the invention in the form of a suspension or a solution, or else can be incorporated into the particles of crosslinked polymer or attached to the polymers (linear and/or crosslinked), for example via a chemical bond.

Of course, those skilled in the art will make sure, on this occasion, that the adjuvant(s) optionally added is (are) compatible with the intrinsic properties associated with the injectable gel-forming composition in accordance with the invention.

A subject of the present invention is also the use of at least one injectable gel-forming composition as described above, for filling pipes and cavities. In particular, the injectable gel-forming composition in accordance with the invention can especially be used for producing therapeutic vascular occlusions (embolizations), for tissue reconstruction or the treatment of gastro-esophageal reflux or urinary incontinence, for percutaneous implantation, or else for reducing wrinkles.

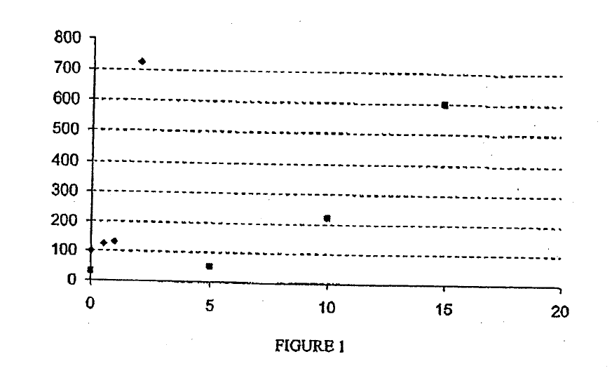

Besides the above provisions, the invention also comprises other provisions which emerge from the following description, which refers to an example of preparation of injectable gel-forming compositions containing a linear p(HEMA) polymer and particles of crosslinked polymer with various degrees of crosslinking, to an example concerning a comparative study of injectable compositions based on linear polymer, possibly containing particles of crosslinked polymer, to an example of production of an arterial embolization in sheep with an injectable composition in accordance with the invention, compared to a composition that is not part of the invention, and also to the attached FIG. 1 which represents the viscosity of injectable compositions based on HPMA-TBA or on HPMA-AAMA as linear polymer, as a function of the concentration of particles of crosslinked polymer.

It should be clearly understood, however, that these examples are given only by way of illustration of the subject of the invention, of which they in no way constitute a limitation.

### EXAMPLE 1

### Preparation of Injectable Gel-Forming Compositions Containing a Polymer p(HEMA) Loaded with Various Particles of Crosslinked Polymer

1) Preparation of a Solution of Linear p(HEMA)

A solution of p(HEMA) (sold under the reference 18894-100 by the company Polysciences Inc. USA) at 12% (m/V) in 96.2% ethanol is prepared.

2) Synthesis and Preparation and Study of Swelling in Water, of Particles of HEMA-Trisacryl® Copolymers

Particles composed of acrylic copolymers (HEMA and/or Trisacryl®) in various proportions and with various degrees of crosslinking are prepared. In the interests of clarity of the description, only the synthesis of particles composed of 50% HEMA and 50% Trisacryl is given in this example. The synthesis of particles having other percentages of each of these two monomers is carried out according to the same process by simply varying the amounts of monomers used. By way of example, for the synthesis of particles composed of 100% HEMA, it is sufficient to use double the amount of HEMA without using Trisacryl®, and vice versa for particles composed of 100% Trisacryl®.

Synthesis and Preparation of Particles

Reagents used:
HEMA: 97% 2-hydroxyethyl methacrylate monomer sold by the company Aldrich under the reference 12,863-5; batch 05808 KI;
Trisacryl®: powdered Trisacryl® monomer sold by the company Biosepra, France, under the reference 5014;
MBA: methylenebisacrylamide (crosslinking agent) sold by the company Biosepra under the reference 7857;
Initiating agent: ammonium peroxodisulfate sold by the company Prolabo under the reference 21300-293;
TMEDA: tetramethylethylenediamine sold by the company Acros Organics.

Synthesis

The synthesis is carried out by radical polymerization in solution.

In order to prepare particles composed of 50% of HEMA and 50% Trisacryl®, 33 ml of distilled water, 5 g of Trisacryl® monomer, 5 ml of HEMA monomer and 100 mg of MBA (i.e. 1% of crosslinking agent m/m) are poured into a first beaker. The mixture obtained is heated at a temperature of approximately 50° C. and stirred in order to facilitate the dissolution of the various reagents. The temperature and the stirring are maintained throughout the duration of the synthesis. Bubbling argon makes it possible to eliminate the O₂ molecules that could hinder the reaction by taking up free radicals.

250 ml of initiating agent are dissolved in 1 ml of deionized water in a second beaker. The solution is magnetically stirred and then poured into the mixture contained in the first beaker. If the reaction does not start after a few minutes, a few drops of TMEDA are added, which also provides initiation of the synthesis.

After a few minutes, the solution solidifies and the stirring and heating are then stopped. The crosslinked polymer obtained, consisting of 50% HEMA and 50% Trisacryl®, is cut up into small fragments and is then put to soak in a large beaker of deionized water. Two successive washes are thus carried out.

Preparation of the Particles

After washing and elimination of the water, the polymer is placed in a crystallizing dish and dried in an oven and then in a vacuum oven.

After drying, the polymer is reduced to powder in an automatic mortar for a few hours. It is then sieved through a 40 μm sieve. The particles of crosslinked polymer are then stored in glass bottles, placed in a desiccator containing a desiccating product.

Study of the Swelling of the Particles of Crosslinked Polymers in Ethanol

For each of the crosslinked polymers prepared, the particle swelling measurements were carried out in graduated cylinders using 1 ml of dry particles.

After the particles have been suspended in 50 ml of ethanol, they are left to sediment. When the sedimentation is stable, the volume of the particles is measured. The ethanol is replaced with 50 ml of water. Homogenization is carried out by means of a Vortex® and by mixing with a Pasteur pipette. These operations are repeated several times (at least three times) in order to eliminate all the ethanol. When the particles have sedimented, the volume of the particles is again measured.

The variation in total volume (τ) (as a percentage) is defined by the following formula:
τ=[(Volume of the particles in water (V_{water})−Volume of the dry particles (V_{dry}))/V_{dry}]×100

The variation in volume (as a percentage) of the dry particles in ethanol (τV1) is calculated using the following formula:
τV1=[(Vₑₜₕₐₙₒₗ−V_{dry})/V_{dry}]×100

The variation in volume (as a percentage) of the particles after passing from ethanol to water (τV2) is calculated using the following formula:
τV2 ((V_{water}−Vₑₜₕₐₙₒₗ)/Vₑₜₕₐₙₒₗ)×100 in which Vₑₜₕₐₙₒₗ is the volume of the particles in ethanol.

The variations in volumes (as a percentage) observed for the various particles prepared are reported in table I below:

TABLE I

| Particle composition (%) | | | | | |
|---|---|---|---|---|---|
| HEMA | Trisacryl ® | MBA | τV1 | τV2 | τ |
| 100 | 0 | 10 | 190 | −17 | 140 |
| 50 | 50 | 10 | 100 | 60 | 220 |
| 0 | 100 | 10 | 20 | 300 | 380 |
| 100 | 0 | 4 | 200 | −20 | 140 |
| 50 | 50 | 4 | 130 | 91 | 340 |
| 0 | 100 | 4 | 30 | 330 | 460 |
| 50 | 50 | 2 | 100 | 110 | 320 |
| 100 | 0 | 1 | 240 | 9 | 210 |
| 50 | 50 | 1 | 130 | 126 | 420 |
| 0 | 100 | 1 | 20 | 550 | 680 |

All these results show that the particles of cross-linked polymer obtained from a solution containing 100% HEMA and 1% crosslinking agent swell a great deal (240%) in ethanol. The particles of crosslinked polymer obtained from a solution containing 50% HEMA and 50% of Trisacryl and the particles consisting of 100% of Trisacryl®, and exhibiting a 1% degree of crosslinking, swell a great deal in water (respectively, 420 and 680%).

3) Preparation of Injectable Compositions Based on p(HEMA) and on Particles of Crosslinked Polymer (Compositions in Accordance with the Invention)

a) Study of the Viscosity of Compositions of Linear p(HEMA) in Ethanol Loaded with Particles of Crosslined p(HEMA), and Comparison with a Solution Not Containing Particles

Compositions of linear p(HEMA) at 12% (m/V) in 96.2° ethanol are prepared according to the protocol described above in 1), containing or not containing 10% (m/V) of particles consisting only of p(HEMA) crosslinked at 1, 4 or 10% (m/V) according to the protocol described above in 2).

The viscosity of the compositions was measured using a Hanke RheoStress® RS 100 controlled-stress rheometer sold by the company Rheo, Champlan, France. A plate/plate geometry was used. The protocol consisted of an increase in shear from 0 to 500 Pa over a period of two minutes at 20° C.

The viscosity of these compositions was compared with that of the solution, prepared above in 1), of p(HEMA) at 12% (m/V) in ethanol and not containing particles of crosslinked polymer.

The results obtained are reported in table II below:

TABLE II

| | Viscosity in |
|---|---|
| Injectable compositions | centipoises (cP) |
| p(HEMA) (12%)* | 100 |
| p(HEMA) (12%) + 10% of particles | 2500 |
| of p(HEMA) crosslinked at 1% | |
| p(HEMA) (12%) + 10% of particles | 1000 |
| of p(HEMA) crosslinked at 4% | |
| p(HEMA) (12%) + 10% of particles | 500 |
| of p(HEMA) crosslinked at 10% | |

*comparative solution that is not part of the invention

These results show that the viscosity of the injectable compositions in accordance with the invention is much higher than that of the solution not containing particles, and that the lower the degree of crosslinking of the polymer forming the particles, the higher the viscosity of the compositions in accordance with the invention.

b) Injection, into Water, of Compositions of p(HEMA) in Ethanol Loaded with Particles of Crosslinked Trisacryl or with Particles of Crosslinked p(HEMA), and Comparison with a Solution of p(HEMA) in Ethanol not Containing Particles of Crosslinked Polymer

Various compositions of linear p(HEMA) at 12% (m/V) in 96.2° ethanol are prepared according to the protocol described above in 1), containing 10% (m/V) of particles consisting only of Trisacryl® crosslinked at 10% (m/V) or 10% (m/V) of particles consisting only of p(HEMA) crosslinked at 10% (m/V), according to the protocol described above in 2).

To do this, the particles of Trisacryl® are weighed out directly in a glass tube or pulled in using a valve. The solution of linear p(HEMA) in ethanol is added with an automatic pipette. The particles were distributed beforehand on the sides of the tubes in order to facilitate homogenization of the suspension with a Vortex®. A magnetic bar is introduced into the suspension. The composition is then left to homogenize by magnetic stirring for 12 hours.

By way of comparison, the behavior, during injection into water, of the solution of linear p(HEMA) at 12% (m/V) in ethanol as prepared above in 1) is also studied.

4) Injection into Water

The injection into water is carried out under the following conditions: 1 ml of each of the compositions thus prepared is injected into a beaker filled with deionized water, by means of a syringe equipped with a 0.6 mm diameter needle. Rotational movement of the syringe in the beaker makes it possible to create a slight shear.

The results obtained are as follows:
Composition of linear p(HEMA) at 12% (m/V) in ethanol containing 10% (m/V) of particles of p(HEMA) crosslinked at 10% (m/V): the particles and the polymer have an affinity for one another, which brings about a large increase in viscosity, but the solidity of the polymer precipitate is not yet sufficient to form a matrix of polymer that traps the particles of p(HEMA) in a sufficiently solid manner to trap the particles of p(HEMA) and withstand a considerable shear. This composition in accordance with the invention is therefore more particularly intended to be used for filling pipes or cavities that are not subjected to too substantial a flow.

By way of comparison:
Injection of the solution of p(HEMA) in ethanol without particles of crosslinked polymer results in the appearance of a very slightly cohesive flaky precipitate. This experiment demonstrates that the linear p(HEMA) cannot be used alone in solution in a water-miscible solvent with the aim of forming a cohesive precipitate.
Composition of linear p(HEMA) at 12% (m/V) in ethanol containing 10% (m/V) of particles of Trisacryl® crosslinked at 10% (m/V): when injected into water, the particles of crosslinked p(HEMA) and the linear p(HEMA) polymer separate, the linear polymer forming relatively noncohesive filaments. The particles and the polymer that have no affinity for one another do not form a cohesive gel.

### EXAMPLE 2

### Preparation of Injectable Compositions Containing Various Copolymers Based on Hydroxypropyl Methacrylate (HPMA) and Particles of Trisacryl®/p(HEMA) (50/50) Copolymers

A) Preparation and Study of Comparative Solutions Comprising Copolymers Based on HPMA Without Particles of Crosslinked Polymer (Solutions Which are Not Part of the Invention)

1) Preparation of HPMA-Based Solutions

a) Synthesis of Copolymers

Reagents Used:

HPMA: 2-hydroxypropyl methacrylate sold by the company Polysciences Inc. under the reference 00730,
AIBN: 2,2′-azobis(2-methylpropionitrile) at 98% sold by the company Acros Organics under the reference 201-132-3,
AN: acrylonitrile sold by the company Aldrich under the reference 11,021-3,
TBA: N-tert-butylacrylamide at 97%, sold by the company Aldrich under the reference 41, 177-9,
AAMA: acetoacetoxyethyl methacrylate sold by the company Acros Organics under the reference 247950250,
Absolute ethanol
96.2% ethanol

Synthesis

The various reagents used in the syntheses and their proportions are indicated in table III below:

TABLE III

| | Monomer | Comonomer | AIBN | Ethanol |
|---|---|---|---|---|
| Copolymer | (ml) | (ml) | (mg) | (ml) |
| HPMA-AN | HPMA: 10 | AN: 3 | 50 | 30 |
| HPMA-TBA | HPMA: 10 | TBA: 2 | 50 | 30 |
| HPMA-AAMA | HPMA: 5 | AAMA: 1.5 | 25 | 15 |

10 ml of HPMA monomer 1 are poured into a 1 liter two-necked round-bottomed flask. 50 mg of AIBN (copolymer-ization-initiating agent) and 30 ml of absolute ethanol, and optionally the amount indicated above of comonomer 2 (AN, TBA or AAMA) are then added.

A condensing column is placed on the round-bottomed flask, a waterbath makes it possible to regulate the temperature of the reaction mixture. An ovoid stirrer makes it possible to homogenize the solution. Argon is bubbled in for 10 minutes in order to eliminate the O₂ molecules that could take up the free radicals and impair the polymerization reaction. When the bubbling is complete, the waterbath is brought to a temperature of between 60 and 70° C. The stirring is maintained for 10 minutes, and then 20 ml of ethanol are subsequently added in order to decrease the viscosity of the mixture. Two successive precipitations are then carried out.

First precipitation: the reaction mixture is poured into a 5 liter beaker containing 4 liters of deionized water. Upon contact with the water, the reaction mixture precipitates and forms a polymer cloud. This cloud is then wound around a glass stirrer in the form of an anchor and removed from the beaker. The polymer is then redissolved in 500 ml of absolute ethanol at a temperature of 70° C. in order to wash from it the impurities that could remain (monomers, initiating agent). The polymer solution is then cooled.

Second precipitation: the solution is then precipitated a second time as described above.

The precipitated polymer recovered is then dried in an oven at a temperature of approximately 50° C. for several hours, and then in a vacuum oven for approximately 12 hours. When the polymer is completely dry, it is broken up into small pieces manually and packaged in hermetically sealed plastic bottles.

2) Measurements of the Viscosity of the Various Polymer Solutions

The polymer solutions are prepared by dissolving each of the polymers prepared above (HPMA-AN, HPMA-TBA and HPMA-AAMA) in a given solvent (absolute ethanol, N-methylpyrrolidone (NMP) or ethyl lactate). The concentrations are established by mass of polymer per volume of solvent (for example: 20%=20 g of polymer per 100 ml of solvent).

The viscosity of the various polymer solutions at various concentrations in the three solvents was measured on a CSL² 100 rheometer sold by the company TA Instruments, USA, using a cone/plate geometry. Each of the solutions tested was subjected to a stepwise increase in stress from 0.5 to 5 Pa, followed by a decrease in stress from 5 to 0.5 Pa. The measurements were carried out at a temperature of 20° C.

The viscosities obtained for each of the polymer solutions tested are reported in table IV below:

TABLE IV

| | Polymer | | | |
|---|---|---|---|---|
| | concentration | Viscosities measured (in cP) | | |
| Solvent | (%) | HPMA-AN | HPMA-TBA | HPMA-AAMA |
| Ethanol | 5 | 5 | 8 | 8 |
| Ethanol | 10 | 22 | 30 | 27 |
| Ethanol | 15 | 72 | 75 | 84 |
| Ethanol | 20 | 202 | 164 | 260 |
| NMP | 5 | 14 | 18 | 17 |
| NMP | 10 | 47 | 62 | 59 |
| NMP | 15 | 109 | 146 | 161 |
| NMP | 20 | 222 | 334 | 371 |
| Ethyl | 5 | 16 | 19 | 25 |
| lactate | | | | |
| Ethyl | 10 | 49 | 65 | 83 |
| lactate | | | | |
| Ethyl | 15 | 140 | 180 | 251 |
| lactate | | | | |
| Ethyl | 20 | 350 | 428 | 767 |
| lactate | | | | |

These results show that, in terms of viscosity, absolute ethanol is the worst solvent for the three polymers studied since it induces the lowest viscosities. However, absolute ethanol exhibits, among the three solvents used in this example, the greatest diffusivity in water, a decisive advantage for the phase separation of an embolization solution.

The solubility parameters of the solvents studied, as given, for example, in the work “Polymer Science Dictionary, Essex (England), Elsevier Science Publishers, 1989, is 26 (MPa)^{1/2} for ethanol, 20 (MPa)^{1/2} for ethyl lactate and 23 (MPa)^{1/2} for NMP. Since water has a parameter of 47 (MPa)^{1/2}, it is ethanol that has the greatest diffusivity in water.

3) Injection into Water

The injection into water of the solutions of these three linear copolymers (HPMA-AN, HPMA-TBA and HPMA-AAMA) at 20% (m/V) in ethanol results in precipitates that are more cohesive than the solution of p(HEMA) not containing particles of crosslinked polymer, prepared and tested above in example 1, but that have a small volume compared with the injected volume, due to the considerable withdrawal of the solvent that occurs during the contact with water.

Consequently, these results show that these three linear copolymers cannot be used alone in solution in a water-miscible solvent with the aim of forming a cohesive precipitate.

B) Preparation and Studies of Compositions Comprising Copolymers Based on HPMA and Particles of Trisacryl®HEMA (Injectable Compositions in Accordance with the Invention)

1) Preparation of Injectable Compositions

Various injectable compositions in accordance with the invention are prepared, containing 10% (m/V) of a copolymer of HPMA-TBA or of HPMA-AAMA, as prepared above in A) 1), and containing:
0.1, 1 or 2%, (m/V) of particles of 50% Trisacryl®—50% HEMA random copolymer, crosslinked at 2% (m/V), as prepared above in example 1, paragraph 2), for the compositions based on HPMA-AAMA copolymer, and
5, 10 or 15% of particles of 50% Trisacryl®—50% HEMA random copolymer, crosslinked at 2% (m/V), as prepared above in example 1, paragraph 2), for the compositions based on HPMA-TBA copolymer.

Two comparative solutions without particles of crosslinked copolymers but containing, respectively, 10% (m/V) of an HPMA-TBA copolymer or 10% (m/V) of HPMA-AAPA were also prepared.

The viscosity of the compositions and comparative solutions thus prepared was measured according to the method described above in this example, paragraph 2), but with a CSL 100 rheometer sold by the company TA Instruments, USA, with a plate/plate geometry.

The viscosities obtained, as a function of the amount of particles contained in each of the compositions, are represented in the attached FIG. 1, in which the viscosity (in mPa·s) is expressed as a function of the concentration of particles (m/V), the black squares representing the viscosity of the compositions based on HPMA-TBA copolymer and the black diamonds representing the viscosity of the compositions based on HPMA-AAMA copolymer.

These results show that the viscosity of the compositions based on HPMA-AAMA copolymer increases much more rapidly, as a function of the concentration of particles, than that of the compositions based on HPMA-TBA copolymer. These results therefore indicate that the interactions between the HPMA-AAMA copolymer and the particles are greater than the interactions between the HPMA-TBA copolymer and the particles.

2) Injection into Water of the Compositions and Solutions of HPMA-Based Copolymers and Containing or not Containing Particles of Trisacryl®-HEMA

Injectable compositions containing or not containing (comparative solutions) particles of 50% Trisacryl®-50% HEMA random copolymer, crosslinked at 2% (m/V), as prepared above in example 1, paragraph 2), are prepared. The composition and the viscosity of the various injectable compositions and comparative solutions prepared are given in detail in table V below:

TABLE V

| Injectable | | | | | |
|---|---|---|---|---|---|
| composi- | Nature | Amount of | Nature | Amount of | |
| tions and | of the | copolymer | of the | particles | Viscosity |
| solutions | copolymer | (% m/V) | solvent | (% m/V) | (mPa · s) |
| 1* | HPMA-AN | 20 | Ethanol | — | 200 |
| 2* | HPMA-AAMA | 20 | Ethanol | — | Nd |
| 3* | HPMA-AAMA | 20 | Ethanol | — | Nd |
| 4* | HPMA-AAMA | 20 | Ethanol | — | Nd |
| 5* | HPMA-AAMA | 20 | Ethanol | — | Nd |
| 6 | HPMA-AAMA | 10 | Ethanol | 2 | 700 |
| 7 | HPMA-AAMA | 10 | Ethanol | 1 | 700 |
| 8 | HPMA-AAMA | 10 | NMP | 1 | Nd |
| 9 | HPMA-AAMA | 10 | NMP | 3 | Nd |
| 10* | HPMA-TBA | 20 | Ethanol | — | 160 |
| 11 | HPMA-TBA | 20 | Ethanol | 10 | Nd |

*Comparative solutions that are not part of the invention

The injection into water of these various compositions and comparative solutions was carried out on a dynamic bench in order to stimulate an embolization. The dynamic bench consists of a reservoir filled with water and located above the height of the site of flow of the water, at constant pressure, in tubing 2 mm in diameter. Each of the compositions or solutions 1 to 11 to be tested is injected, with a 5 ml syringe, into the tubing through a needle with an inner diameter of 0.3 mm and an outer diameter of 4.1 mm, that passes through the wall of the tubing and the end of which is placed at the center of the tubing. The dimensions of the bench (length of the needle, distance between the end of the needle and the water inlet, etc.) are chosen first to obtain a laminar flow in the tubing. The average speed of the water in the tubing is 60±5 cm/second. A beaker and a balance make it possible to measure the flow rate in the tubing. The flow rate is regulated by decreasing the lumen of the water inlet in the tubing upstream of the injecting needle, using one or two Mohr clamps. A video camera connected to a laptop computer makes it possible to film the injections in order to compare and evaluate the various gels formed when the compositions and solutions are injected into the water. A scale graduated in centimeters, positioned opposite the tubing, and the zero of which is aligned on the end of the injection needle located at the center of the tubing, makes it possible to measure the length of the embolus formed after injection.

The results obtained for each of the injectable compositions or comparative solutions 1 to 11 were formulated in the form of a composite score which makes it possible to characterize the embolization capacity of each of the compositions and to compare them with one another. This score is made up of the following three elements:
the cohesion of the injected composition when it solidifies upon contact with the water: it is assessed either as a function of the fragmentation or nonfragmentation of the injected composition, or based on its ability to withstand the flow;
the degree of occlusion with respect to the diameter of the tube: it corresponds to the percentage of the lumen of the tube that is occluded by the composition or solution injected;
the length of the embolus: it quantifies the rapidity of occlusion of the lumen of the bench; the shorter the embolus enabling the occlusion, the more effective the embolization composition.

As regards the criterion of cohesion of the composition or solution injected, the grading is performed in the following manner:
the gel fragments as soon as it leaves the needle: +
the gel stretches out in the stream, and then fragments: ++
the gel stretches out in the stream without fragmenting: +++
the gel is virtually immobile, it barely advances in the stream: ++++.

As regards the degree of occlusion, the grading is performed in the following manner:
occlusion less than 33%: +
occlusion between 33 and 66%: ++
occlusion between 66% and 99%: +++
complete occlusion: ++++
complete occlusion with reflux toward the needle: +++++.

As regards the length of the embolus, and in the event of occlusion, the grading is performed in the following manner:
length of the embolus greater than 3 cm: +
length of the embolus between 3 and 2 cm: ++
length of the embolus less than 1 cm: +++.

The total of the number of pluses obtained for each of the criteria studied results in an overall grade, which is the composite score, for which the maximum possible is 12.

The results obtained for each of the injectable compositions or comparative solutions 1 to 11 are reported in table VI below:

TABLE VI

| Injectable | | | | |
|---|---|---|---|---|
| compositions | | Degree of | Length of | Composite |
| or solutions | Cohesion | occlusion | the embolus | score |
| 1* | + | + | not formed | 2 |
| 2* | ++++ | ++++ | + | 9 |
| 3* | + | + | not formed | 2 |
| 4* | ++++ | ++ | not formed | 6 |
| 5* | ++ | ++ | not formed | 4 |
| 6 | ++ | + | not formed | 3 |
| 7 | ++ | ++ | not formed | 4 |
| 8 | ++ | + | not formed | 3 |
| 9 | ++ | + | not formed | 3 |
| 10* | ++++ | ++++ | + | 9 |
| 11 | ++++ | +++++ | ++ | 11 |

*Comparative solutions that are not part of the invention

These results show that the injectable comparative solution no. 10, based on HPMA-TBA copolymer without particles of crosslinked polymer, produced better results in terms of embolization than the injectable solutions based on the HPMA-AN or HPMA-AAMA copolymers.

In addition, the addition of particles of 50% Trisacryl®-50% HEMA random polymers, at 2% cross-linking (m/V), to this solution based on HPMA-TBA copolymer at 20% in ethanol (composition no. 11) improves the embolization capacity of the composition no. 10 not containing said particles.

3) Study of the Swelling in Water of an Injectable Composition in Accordance with the Invention

1 ml of an injectable composition of HPMA-TBA at 10% (m/v) in ethanol, containing 15% (m/v) of particles of Trisacryl®/HEMA (50/50) crosslinked at 2% (m/V), is taken up in a syringe.

This composition is injected into a beaker filled with water, with magnetic stirring, so as to form an embolus that is stable and sufficiently compact to be handled. When the embolus is formed, it is left to stand in the beaker with slow magnetic stirring. After about fifteen hours, the white and firm appearance of the embolus implies that the solvent exchanges in the sample are complete. The embolus is then removed and placed in a graduated cylinder containing a precise volume of water. The difference in volume observed after introduction of the embolus into the cylinder corresponds to the volume of the embolus.

In this example, the volume of the embolus is 1.6 ml.

This result shows that, after injection into water and solidification of the linear polymer matrix, the particles swell and induce a swelling of the gel of more than 60%.

### EXAMPLE 3

### Production of Arterial Embolizations in Sheep with an Injectable Composition in Accordance with the Invention and Comparison with a Solution not Containing Particles of Crosslinked Polymer

The aim of this example is to compare the effects obtained, in terms of arterial embolization in sheep, with injectable compositions in accordance with the invention, i.e. comprising particles of crosslinked polymer, and injectable solutions not in accordance with the invention, i.e. not comprising particles of crosslinked polymer.

To do this, the injectable solution no. 10 and the injectable composition no. 11 as described above in table V of example 2 were used.

Before use, the solution and the composition are sterilized at a temperature of 70° C.

Each of them is then made radio-opaque by the addition and the mixing in of a micronized powder of tantalum at a dose of 0.5 g per ml of composition.

The injection of these preparations was carried out in sheep, under general anesthetic: entry was made through the femoral artery with a 5F introductory catheter, and then several intercostal arteries were catheterized selectively. 0.5 ml of gel-forming composition is injected into each of them, under fluoroscopy. The distribution of the gel is monitored under a fluoroscope for several minutes, and then checked at regular intervals for one hour.

The solution not in accordance with the invention, i.e. not containing particles of crosslinked polymer, advances in the vascular tree, pushed by the arterial flow, over several centimeters during the first minutes following injection. The occlusion finally obtained is incomplete, distal and unstable.

On the other hand, the composition in accordance with the invention, i.e. containing particles of crosslinked polymer in suspension, advances very little in the flow and remains localized in the form of gel at the end of the catheter. The occlusion obtained is complete, proximal and stable. The radiographic verification carried out one hour after injection shows that the solution not containing the particles of crosslinked polymer results in gel fragment formation over the entire path of the artery, whereas injection of the composition in accordance with the invention results in the formation of a proximal embolus that remains stable and in place.

1. A polymer-based injectable gel-forming composition for intratissue and/or intravascular implantation, characterized in that it comprises:
at least one linear polymer that is water-insoluble and soluble in at least one water-miscible solvent,
at least one water-insoluble, hydrophilic crosslinked polymer, and
at least one biocompatible, water-miscible solvent;
and in that it is in the form of a suspension of particles of said hydrophilic crosslinked polymer in a solution of said linear polymer.

2. The injectable gel-forming composition as claimed in claim 1, characterized in that the linear polymer(s) is (are) chosen from poly(alkyl acrylates), poly(alkyl methacrylates), poly(alkyl cyanoacrylates), poly(vinyl acetates), poly(vinyl butyrates), poly(vinyl formals), poly(vinyl acetals), poly(vinyl butyrals), polyoxypropylenes, polyoxytetramethylenes, water-insoluble cellulose esters, water-insoluble esters of chitosan or other polysaccharides, polylactides, polyglycolides, polycaprolactone, poly(malic acid) esters, poly(maleic acid) esters, poly(fumaric acid) esters, and water-insoluble linear copolymers or derivatives comprising these compounds.

3. The injectable gel-forming composition as claimed in claim 2, characterized in that the linear polymer(s) is (are) chosen from poly(hydroxyethyl methacrylate), poly(methyl methacrylate), poly(hydroxypropyl meth-acrylate), copolymers of hydroxyethyl methacrylate or hydroxypropyl methacrylate and of acrylonitrile, copolymers of hydroxyethyl methacrylate or hydroxy-propyl methacrylate and of N-tert-butylacrylamide, copolymers of hydroxyethyl methacrylate or hydroxypropyl methacrylate and of acetoacetoxyethyl methacrylate, poly(N-acryloyl-2-amino-2-hydroxymethyl-1,3-propanediol), poly(n-2-hydroxypropyl methacrylamide), and derivatives thereof.

4. The injectable gel-forming composition as claimed in claim 3, characterized in that the linear polymer(s) is (are) chosen from copolymers of hydroxypropyl methacrylate and of acrylonitrile, copolymers of hydroxypropyl methacrylate and of N-tert-butylacrylamide and copolymers of hydroxypropyl methacrylate and of acetoacetoxyethyl methacrylate.

5. The injectable gel-forming composition as claimed in any one of the preceding claims, characterized in that the linear polymer(s) represent(s) from 3 to 25% (m/V).

6. The injectable gel-forming composition as claimed in any one of the preceding claims, characterized in that the hydrophilic crosslinked polymer(s) is (are) chosen from the polymers derived from the crosslinking of the water-insoluble linear polymers as defined in claim 2.

7. The injectable gel-forming composition as claimed in any one of claims 1 to 5, characterized in that the hydrophilic crosslinked polymer(s) is (are) chosen from the polymers derived from the crosslinking of water-soluble linear polymers.

8. The injectable gel-forming composition as claimed in claim 7, characterized in that the water-soluble linear polymers are chosen from alginates; starch derivatives; cellulose ethers; cellulose acetates with a degree of substitution of between 0.6 and 0.8; cellulose sulfates; water-soluble polysaccharides; chitosan salts; acrylic and methacrylic polymers; substituted or unsubstituted polyacrylamides and polymethacrylamides; hydrolyzed derivatives of poly(vinyl acetates); polymers derived from polyoxyethylene, polyethyleneimine; soluble salts of polyvinylpyridine; polyvinylpyrrolidone; polyurethanes; salts thereof and copolymers thereof.

9. The injectable gel-forming composition as claimed in claim 6, characterized in that the crosslinked polymer(s) is (are) chosen from the crosslinked polymers of hydroxyethyl methacrylate, of hydroxypropyl methacrylate or of poly(N-acryloyl-2-amino-2-hydroxy-methyl-1,3-propanediol), and also from the crosslinked copolymers of hydroxyethyl methacrylate and of poly(N-acryloyl-2-amino-2-hydroxymethyl-1,3-propanediol), or of hydroxypropyl methacrylate and of poly(N-acryloyl-2-amino-2-hydroxymethyl-1,3-propanediol).

10. The injectable gel-forming composition as claimed in any one of the preceding claims, characterized in that the degree of crosslinking of the crosslinked polymer is between 0.5 and 12% (m/V).

11. The injectable gel-forming composition as claimed in any one of the preceding claims, characterized in that the crosslinked polymer(s) represent(s) from 1 to 30% (m/V).

12. The injectable gel-forming composition according to any one of the preceding claims, characterized in that the size of the particles of crosslinked polymer(s) is between 1 and 1000 μm.

13. The injectable gel-forming composition as claimed in any one of the preceding claims, characterized in that it comprises at least one linear hydroxyethyl methacrylate or hydroxypropyl methacrylate polymer or a linear hydroxyethyl methacrylate-based or hydroxypropyl methacrylate-based copolymer and particles of cross-linked polymers of hydroxyethyl methacrylate, of hydroxypropyl methacrylate or of poly(N-acryloyl-2-amino-2-hydroxymethyl-1,3-propanediol), and/or of crosslinked copolymers of hydroxyethyl methacrylate and of poly(N-acryloyl-2-amino-2-hydroxymethyl-1,3-propanediol) or of hydroxypropyl methacrylate and of poly(N-acryloyl-2-amino-2-hydroxymethyl-1,3-propanediol).

14. The injectable gel-forming composition as claimed in claim 13, characterized in that it comprises:
at least one linear, hydroxyethyl methacrylate-based or hydroxypropyl methacrylate-based copolymer, and
at least particles of crosslinked copolymers of hydroxyethyl methacrylate or hydroxypropyl methacrylate and of poly(N-acryloyl-2-amino-2-hydroxymethyl-1,3-propanediol).

15. The injectable gel-forming composition as claimed in any one of the preceding claims, characterized in that the biocompatible, water-miscible solvent(s) is (are) chosen from N-methylpyrrolidone, dimethylethylamide, diethylene glycol dimethyl ether, ethyl lactate, ethanol, dimethoxyethane, dimethylsulfoxide, glycofurol, and mixtures thereof.

16. The injectable gel-forming composition as claimed in claim 15, characterized in that said solvents are chosen from ethanol and N-methylpyrrolidone.

17. The injectable gel-forming composition as claimed in any one of the preceding claims, characterized in that it also contains one or more adjuvants chosen from dyes; imaging markers; anti-inflammatory agents; angiogenic agents; antimitotics; angiogenesis inhibitors; growth factors; vitamins; hormones; proteins; vaccines; peptides; antiseptics and antimicrobial agents.

18. The use of at least one injectable gel-forming composition as defined in any one of claims 1 to 17, for filling pipes and cavities.

19. The use of at least one injectable gel-forming composition as defined in any one of claims 1 to 17, for producing therapeutic vascular occlusions, for tissue reconstruction or the treatment of gastro-esophageal reflux or urinary incontinence, for percutaneous implantation, or for reducing wrinkles.

20. A linear copolymer-based intermediate solution, characterized in that it comprises:
at least one linear copolymer of hydroxypropyl methacrylate and of acrylonitrile, and/or at least one copolymer of hydroxypropyl methacrylate and of N-tert-butylacrylamide and/or at least one copolymer of hydroxypropyl methacrylate and of acetoacetoxyethyl methacrylate, and
at least one biocompatible, water-miscible solvent;
it being understood that, when said intermediate solution contains a linear copolymer of hydroxypropyl methacrylate and of acrylonitrile, then the solvent is not dimethyl sulfoxide.
